(19) United States
(12) Patent Application Publication
(10) Pub. No.: US 2024/0082280 A1
(43) Pub. Date: Mar. 14, 2024

(54) COMPOSITION FOR SKIN TREATMENT AND METHODS

(71) Applicant: DERMAZEN, LLC, Carlsbad, CA (US)

(72) Inventor: Austin Beals, Carlsbad, CA (US)

(21) Appl. No.: 18/463,663

(22) Filed: Sep. 8, 2023

Related U.S. Application Data
(60) Provisional application No. 63/405,023, filed on Sep. 9, 2022.

(51) Int. Cl. A61K 31/7004; A61K 33/04; A61K 33/38; A61K 35/08; A61K 36/61; A61K 36/752; A61K 36/886

(52) U.S. Cl. CPC A61K 31/7004; A61K 33/04; A61K 33/38; A61K 35/08; A61K 36/61; A61K 36/752; A61K 36/886

(57) ABSTRACT

A composition for treating skin of a patient treatment. The composition has an overall weight. The composition includes a biofilm neutralizer, an antifungal, and a skin soother. The biofilm neutralizer can include one of a sugar alcohol, Xylitol, and colloidal silver. The antifungal can include one of grapefruit seed extract, sea salt, and tea tree oil. The skin soother can include one of aloe vera, methylsulfonylmethane, niacinamide, hyaluronic acid, rosemary essential oil, and urea, the oils only having a fatty acid chain length of less than C12 or greater than C24. The composition can include purified water comprising greater than approximately 50 percent of the overall weight and less than 60 percent of the overall weight.

### RELATED APPLICATION

This application claims priority on U.S. Provisional Application Ser. No. 63/405,023, filed on Sep. 9, 2022, and entitled, “COMPOSITION FOR SKIN TREATMENT AND METHODS.” As far as permitted, the contents of U.S. Provisional Application Ser. No. 63/405,023 are incorporated in their entirety herein by reference.

### BACKGROUND

Malassezia fungus (the cause of seborrheic dermatitis, dandruff, and most forms of fungal acne) forms a biofilm shield that protects the fungus from antifungal treatment. The fungus also feeds on most oils commonly found in skincare products.

The genus of fungus known as Malassezia causes a variety of skin conditions—specifically seborrheic dermatitis, dandruff, and fungal acne. The fungus is strengthened in the presence of most oils (especially lipids with specific fatty acid chain lengths). The fungus also creates a biofilm layer that protects itself from most antifungal treatments. Most people suffering from these conditions are not aware of the biofilm nor the importance of avoiding inflammatory oils. For this reason, many people fail to find relief from their skin disorder, even after following the guidance and prescriptions of their dermatologist.

Many dermatologists attempt to eliminate the Malassezia fungus (and its related skin conditions) using strong antifungal compounds. These treatments will often provide short-term or limited relief for patients. Unfortunately, these chemicals focus on attacking the fungus without addressing the biofilm layer protecting the yeast from antifungal treatment. Conventional solutions fail to weaken the fungal biofilm while simultaneously strengthening the fungus due to the presence of inflammatory oils. If past treatments have failed, it is likely due to the strength of the fungal biofilm and the presence of oils with a specific fatty acid chain length.

### SUMMARY

The present invention is directed toward a composition for treating a patient's skin. The composition includes a biofilm neutralizer, an antifungal, and a skin soother. The biofilm neutralizer can include a sugar alcohol. The antifungal can include grapefruit seed extract. The skin soother can include aloe vera and an oil. The oil can only have a fatty acid chain length of less than C12 or greater than C24.

In some embodiments, the sugar alcohol is Xylitol.

In certain embodiments, the biofilm neutralizer further includes colloidal silver.

In various embodiments, the antifungal further includes at least one of grapefruit seed extract, sea salt, tea tree oil, and colloidal silver.

In some embodiments, the skin soother further includes at least one of methylsulfonylmethane, niacinamide, hyaluronic acid, and urea.

In certain embodiments, the oil includes an essential oil.

In various embodiments, the skin soother further includes Allantoin.

In some embodiments, the composition further includes purified water comprising greater than approximately 50 percent of the overall weight and less than 60 percent of the overall weight.

The present invention is also directed toward a composition for treating a patient's skin. The composition has an overall weight. The composition includes a biofilm neutralizer, an antifungal, and a skin soother. The biofilm neutralizer includes a sugar alcohol, the sugar alcohol including Xylitol comprising greater than approximately 0.001 percent of the overall weight and less than 90.0 percent of the overall weight. The antifungal includes grapefruit seed extract and colloidal silver. The skin soother includes aloe vera, methylsulfonylmethane, and an oil. The oil only has a fatty acid chain length of less than C12 or greater than C24. The aloe vera comprises greater than approximately 0.001 percent of the overall weight and less than 90.0 percent of the overall weight.

In certain embodiments, the antifungal further includes at least one of sea salt and an essential oil.

In various embodiments, the skin soother further includes at least one of niacinamide, hyaluronic acid, and urea.

In some embodiments, the oil includes an essential oil.

In certain embodiments, the skin soother further includes Allantoin.

In various embodiments, the composition further includes purified water comprising greater than approximately 50 percent of the overall weight and less than 60 percent of the overall weight.

In some embodiments, the colloidal silver comprises greater than approximately 7 percent of the overall weight and less than 12 percent of the overall weight.

In certain embodiments, the methylsulfonylmethane comprises greater than approximately 4 percent of the overall weight and less than 7 percent of the overall weight.

In various embodiments, the grapefruit seed extract comprises greater than approximately 0.1 percent of the overall weight and less than 2.0 percent of the overall weight.

In some embodiments, the oil comprises greater than approximately 0.01 percent of the overall weight and less than 0.11 percent of the overall weight.

The present invention is further directed toward a method of manufacturing the embodiments of the composition disclosed herein.

The present invention is still further directed toward a composition for treating a patient's skin. The composition includes a biofilm neutralizer, an antifungal, and a skin soother.

The biofilm neutralizer can include Xylitol and colloidal silver. The antifungal can include grapefruit seed extract. The skin soother can include grapefruit seed extract, sea salt, and tea tree oil. The oil can only have a fatty acid chain length of less than C12 or greater than C24.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE DRAWING

The novel features of this invention, as well as the invention itself, both as to its structure and its operation, will be best understood from the accompanying figure, taken in conjunction with the accompanying description, in which similar reference characters refer to similar parts, and in which:

FIG. 1 is a flow chart showing one embodiment of a method for treating Malassezia on the skin of a patient;

FIG. 2 is a flow chart showing one embodiment of a method for manufacturing a composition for treating the skin of a patient; and

FIG. 3 is a flow chart showing yet another embodiment of a method for manufacturing a composition for treating the skin of a patient.

While embodiments of the present invention are susceptible to various modifications and alternative forms, specifics thereof, have been shown by way of examples and drawings, and are described in detail herein. It is understood, however, that the scope herein is not limited to the particular embodiments described. On the contrary, the intention is to cover modifications, equivalents, and alternatives falling within the spirit and scope herein.

### DESCRIPTION

Embodiments of the present invention are described herein in the context of a composition for skin treatment and related methods that can be effectively utilized to treat a variety of skin diseases, such as seborrheic dermatitis, dandruff, and fungal acne. Those of ordinary skill in the art will realize that the following detailed description of the present invention is illustrative only and is not intended to be in any way limiting. Other embodiments of the present invention will readily suggest themselves to such skilled persons having the benefit of this disclosure. Reference will now be made in detail to implementations of the present invention, as illustrated in the accompanying drawings. The same or similar nomenclature and/or reference indicators will be used throughout the drawings, and the following detailed description to refer to the same or like parts.

In the interest of clarity, not all of the routine features of the implementations described herein are shown and described. It will, of course, be appreciated that in the development of any such actual implementation, numerous implementation-specific decisions must be made in order to achieve the developer's specific goals, such as compliance with application-related and business-related constraints, and that these specific goals will vary from one implementation to another and from one developer to another. Moreover, it will be appreciated that such a development effort might be complex and time-consuming. However, it would nevertheless be a routine undertaking of engineering for those of ordinary skill in the art having the benefit of this disclosure.

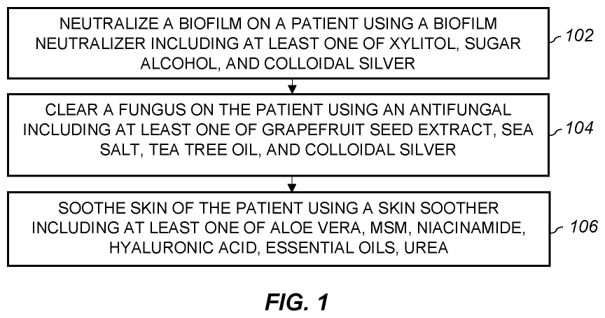

FIG. 1 is a flow chart showing one embodiment of a method for treating Malassezia on the skin of a patient. Although the method is described herein primarily in its potential use with humans, it is appreciated that the method can also be utilized to treat Malassezia on the skin of animals. As used herein, “patient” can include any suitable organism that can receive suitable treatment via compositions described herein.

It is understood that the various steps described herein can be modified as necessary in treating Malassezia on a patient's skin. Additionally, it should also be appreciated that in certain applications, the order of the steps can be modified, certain steps can be omitted, and/or additional steps can be added without limiting the intended scope and breadth of the present invention.

At step 102, a biofilm on the skin of the patient is neutralized using a biofilm neutralizer. The biofilm neutralizer can include any suitable embodiment of the compositions described herein. In some embodiments, the biofilm neutralizer can include Xylitol or another suitable sugar alcohol. Xylitol can weaken the biofilm on the skin of the patient. The composition, including Xylitol, can also inhibit future biofilm growth on the patient's skin. The composition can also include Colloidal Silver, which disrupts the biofilm and clears away fungus. The composition can purposefully exclude oils that strengthen any fungus that may exist on the patient's skin, such as those with fatty acid chain lengths greater than or equal to C12.

At step 104, a fungus on the skin of the patient is cleared using an antifungal. After removing the biofilm in step 102, the antifungal can eliminate the fungus that may exist on the patient's skin. The antifungal can include skin-safe antifungals such as Grapefruit Seed Extract, Tea Tree Essential Oil, Dead Sea Salt, Rosemary Essential Oil, and/or Colloidal Silver.

At step 106, the skin of the patient is soothed using a skin soother. The skin is soothed and nourished without using lipids (such as coconut oil) that feed fungi such as Malassezia. The composition can include soothing ingredients such as Concentrated Aloe Vera, Tea Tree Essential Oil, Dead Sea Salt, Rosemary Essential Oil, Hyaluronic Acid, Niacinamide, Methylsulfonylmethane, Urea, and/or Purified Water.

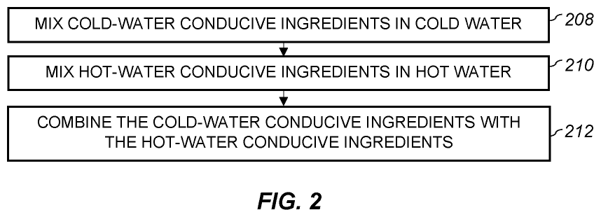

FIG. 2 is a flow chart showing one embodiment of a method for manufacturing a composition for treating the skin of a patient. It is understood that the various steps described herein can be modified as necessary in the process of manufacturing a composition for treating the skin of a patient. Additionally, it should also be appreciated that in certain applications, the order of the steps can be modified, certain steps can be omitted, and/or additional steps can be added without limiting the intended scope and breadth of the present invention.

At step 208, cold-water conducive ingredients are mixed in cold water. As used herein, “cold water” is understood to mean greater than approximately 32 degrees Fahrenheit and less than approximately 75 degrees Fahrenheit. The cold-water conducive ingredients can be mixed for any suitable amount of time. In some embodiments, the cold-water conducive ingredients can be mixed in cold water for greater than approximately 2 minutes and less than approximately 20 minutes. The cold-water conducive ingredients can include at least one of aloe vera concentrated flakes, hyaluronic acid, grapefruit seed extract, tea tree essential oil, and rosemary essential oil.

At step 210, hot-water conducive ingredients are mixed in hot water. As used herein, “hot water” is understood to mean greater than approximately 75 degrees Fahrenheit and less than approximately 150 degrees Fahrenheit. The hot-water conducive ingredients can be mixed for any suitable amount of time. In some embodiments, the hot-water conducive ingredients can be mixed in hot water for greater than approximately 1 minute and less than approximately 10 minutes. The hot-water conducive ingredients can include at least one of Xylitol, sea salt, Colloidal Silver, Niacinamide (vitamin B3), Urea, and Methylsulfonylmethane.

At step 212, the cold-water conducive ingredients are combined with the hot-water conducive ingredients. The cold-water conducive ingredients and the hot-water conducive ingredients can be mixed for any suitable amount of time. In some embodiments, the cold-water conducive ingredients can be combined with the hot-water conducive ingredients for greater than approximately 1 minute and less than approximately 5 minutes.

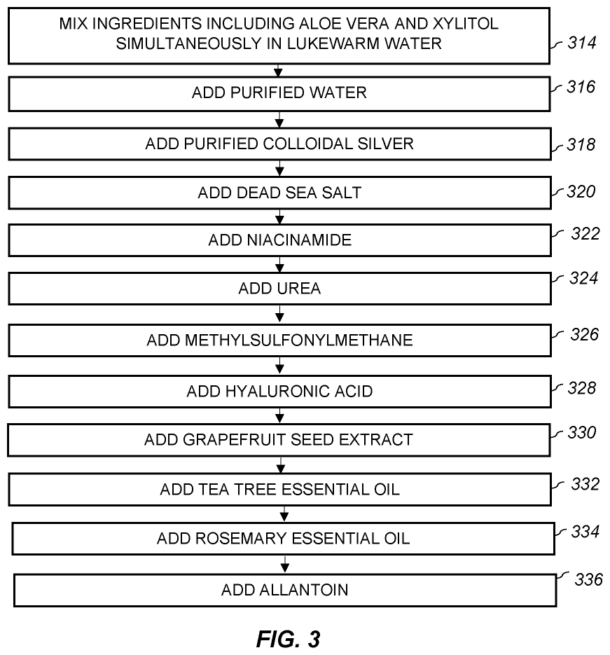

It is also appreciated that the methods of manufacturing described herein can be accomplished with one, constant temperature of water. For example, in some embodiments, a constant temperature of greater than 60 degrees Fahrenheit and less than 100 degrees Fahrenheit can be used to mix all of the ingredients. This range of water temperature can be considered as “lukewarm” for purposes of this disclosure, such as described with respect to FIG. 3.

FIG. 3 is a flow chart showing yet another embodiment of a method for manufacturing a composition for treating the skin of a patient. It is understood that the various steps described herein can be modified as necessary in the process of manufacturing a composition for treating the skin of a patient. Additionally, it should also be appreciated that in certain applications, the order of the steps can be modified, certain steps can be omitted, and/or additional steps can be added without limiting the intended scope and breadth of the present invention.

In various embodiments, the composition may have an overall weight. The percentage of the overall weight of the composition that is formed from its individual components can be varied to suit the specific requirements of the composition. For example, in some embodiments, the purified water can comprise greater than approximately 50% and less than approximately 60% of the overall weight of the composition. More particularly, in various alternative embodiments, the purified water can include at least approximately 60%, 65%, 70%, 75%, 80%, 85%, or 90% of the overall weight of the composition. Alternatively, the purified water can include greater than 90% or less than 60% of the overall weight of the composition.

Additionally, in certain embodiments, the composition may have an overall volume. The percentage of the overall volume of the consumable that is formed from the purified water can also be varied to suit the specific requirements of the composition. For example, in some embodiments, the purified water can comprise greater than approximately 75% and less than approximately 95% of the overall volume of the composition. More particularly, in various alternative embodiments, the base portion can include at least approximately 75%, 80%, 85%, 90%, or 95% of the overall volume of the composition. Alternatively, the purified water can include greater than approximately 95% or less than approximately 75% of the overall volume of the consumable.

In step 314, ingredients including Aloe Vera and Xylitol are mixed simultaneously in lukewarm water. The Aloe Vera can comprise greater than approximately 0.001% of the overall weight and less than 90.0% of the overall weight. The Xylitol can comprise greater than approximately 0.001% of the overall weight and less than 90.0% of the overall weight. The percentage of the overall weight of the composition that is formed from the Aloe Vera and/or the Xylitol can be varied to suit the specific requirements of the composition. For example, in some embodiments, the Aloe Vera and/or the Xylitol can comprise greater than approximately 0.001% and less than approximately 90% of the overall weight of the composition. More particularly, in various alternative embodiments, the Aloe Vera and/or the Xylitol can include at least approximately 60%, 65%, 70%, 75%, 80%, 85%, or 90% of the overall weight of the composition. Alternatively, the Aloe Vera and/or the Xylitol can include greater than 90% or less than 0.001% of the overall weight of the composition.

At step 316, purified water is added to the Aloe Vera and Xylitol combination. In various embodiments, such as described herein, the purified water can comprise greater than approximately 50 percent of the overall weight and less than 60 percent of the overall weight.

At step 318, colloidal silver is added to the combination. The percentage of the overall weight of the composition that is formed from the colloidal silver can be varied to suit the specific requirements of the composition. For example, in some embodiments, the colloidal silver can comprise greater than approximately 7% and less than approximately 12% of the overall weight of the composition. More particularly, in various alternative embodiments, the colloidal silver can include at least approximately 10%, 15%, 20%, 25%, 30%, 35%, or 40% of the overall weight of the composition. Alternatively, the colloidal silver can include greater than 40% or less than 10% of the overall weight of the composition.

At step 320, Dead Sea Salt is added to the combination. The percentage of the overall weight of the composition that is formed from the Dead Sea Salt can be varied to suit the specific requirements of the composition. For example, in some embodiments, the Dead Sea Salt can comprise greater than approximately 8% and less than approximately 13% of the overall weight of the composition. More particularly, in various alternative embodiments, the Dead Sea Salt can include at least approximately 10%, 15%, 20%, 25%, 30%, 35%, or 40% of the overall weight of the composition. Alternatively, the Dead Sea Salt can include greater than 40% or less than 10% of the overall weight of the composition.

At step 322, niacinamide is added to the combination. The percentage of the overall weight of the composition that is formed from the niacinamide can be varied to suit the specific requirements of the composition. For example, in some embodiments, the niacinamide can comprise greater than approximately 1% and less than approximately 5% of the overall weight of the composition. More particularly, in various alternative embodiments, the niacinamide can include at least approximately 5%, 10%, 15%, 20%, 25%, 30%, 35%, or 40% of the overall weight of the composition. Alternatively, the niacinamide can include greater than 40% or less than 5% of the overall weight of the composition.

At step 324, urea is added to the combination. The percentage of the overall weight of the composition that is formed from the urea can be varied to suit the specific requirements of the composition. For example, in some embodiments, the urea can comprise greater than or equal to 0% and less than approximately 6% of the overall weight of the composition. More particularly, in various alternative embodiments, the urea can include at least approximately 10%, 15%, 20%, 25%, 30%, 35%, or 40% of the overall weight of the composition. Alternatively, the urea can include greater than 40% or less than 10% of the overall weight of the composition.

At step 326, methylsulfonylmethane is added to the combination. The percentage of the overall weight of the composition that is formed from the methylsulfonylmethane can be varied to suit the specific requirements of the composition. For example, in some embodiments, the methylsulfonylmethane can comprise greater than approximately 4% and less than approximately 7% of the overall weight of the composition. More particularly, in various alternative embodiments, the methylsulfonylmethane can include at least approximately 10%, 15%, 20%, 25%, 30%, 35%, or 40% of the overall weight of the composition. Alternatively, the methylsulfonylmethane can include greater than 40% or less than 10% of the overall weight of the composition.

A step 328, Hyaluronic Acid is added to the combination. The percentage of the overall weight of the composition that is formed from the Hyaluronic Acid can be varied to suit the specific requirements of the composition. For example, in some embodiments, the Hyaluronic Acid can comprise greater than approximately 0.1% and less than approximately 2% of the overall weight of the composition. More particularly, in various alternative embodiments, the Hyaluronic Acid can include at least approximately 5%, 10%, 15%, 20%, 25%, 30%, 35%, or 40% of the overall weight of the composition. Alternatively, the Hyaluronic Acid can include greater than 40% or less than 5% of the overall weight of the composition.

At step 330, Grapefruit Seed Extract is added to the combination. The percentage of the overall weight of the composition that is formed from the Grapefruit Seed Extract can be varied to suit the specific requirements of the composition. For example, in some embodiments, the Grapefruit Seed Extract can comprise greater than approximately 0.1% and less than approximately 2% of the overall weight of the composition. More particularly, in various alternative embodiments, the Grapefruit Seed Extract can include at least approximately 5%, 10%, 15%, 20%, 25%, 30%, 35%, or 40% of the overall weight of the composition. Alternatively, the Grapefruit Seed Extract can include greater than 40% or less than 5% of the overall weight of the composition.

At step 332, Tea Tree Essential Oil is added to the combination. The percentage of the overall weight of the composition that is formed from the Tea Tree Essential Oil can be varied to suit the specific requirements of the composition. For example, in some embodiments, the Tea Tree Essential Oil can comprise greater than approximately 0.01% and less than approximately 0.11% of the overall weight of the composition. More particularly, in various alternative embodiments, the Tea Tree Essential Oil can include at least approximately 1%, 5%, 10%, 15%, 20%, 25%, 30%, 35%, or 40% of the overall weight of the composition. Alternatively, the Tea Tree Essential Oil can include greater than 40% or less than 1% of the overall weight of the composition.

At step 334, Rosemary Essential Oil is added to the combination. The percentage of the overall weight of the composition that is formed from the Rosemary Essential Oil can be varied to suit the specific requirements of the composition. For example, in some embodiments, the Rosemary Essential Oil can comprise greater than approximately 0.01% and less than approximately 0.11% of the overall weight of the composition. More particularly, in various alternative embodiments, the Rosemary Essential Oil can include at least approximately 1%, 5%, 10%, 15%, 20%, 25%, 30%, 35%, or 40% of the overall weight of the composition. Alternatively, the Rosemary Essential Oil can include greater than 40% or less than 1% of the overall weight of the composition.

At step 336, Allantoin is added to the combination. The percentage of the overall weight of the composition that is formed from the Allantoin can be varied to suit the specific requirements of the composition. For example, in some embodiments, the Allantoin can comprise greater than or equal to 0% and less than approximately 6% of the overall weight of the composition. More particularly, in various alternative embodiments, the Allantoin can include at least approximately 10%, 15%, 20%, 25%, 30%, 35%, or 40% of the overall weight of the composition. Alternatively, the Allantoin can include greater than 40% or less than 10% of the overall weight of the composition.

The composition disclosed herein is the only known treatment for skin using Xylitol to weaken fungal biofilms while simultaneously excluding oils with a fatty acid chain length between C12-C134. Malassezia fungus (the cause of seborrheic dermatitis, dandruff, and most forms of fungal acne) forms a biofilm shield that protects the fungus from antifungal treatment. This biofilm shield can be safely neutralized by the sugar alcohol known as Xylitol. The fungus also feeds on most oils commonly found in skincare products. The composition disclosed herein offers unique relief to treatment-resistant patients by avoiding problematic oils and weakening the biofilm with Xylitol.

The composition disclosed herein can be used topically. In certain embodiments, the composition can be topically applied to the scalp, face, and/or body of the patient. The composition can reduce flakes, calm redness, soothe itching, and eliminate fungi.

It is understood that although a number of different embodiments of the composition (and method for making thereof) have been illustrated and described herein, one or more features of any one embodiment can be combined with one or more features of one or more of the other embodiments, provided that such combination satisfies the intent of the present invention.

While a number of exemplary aspects and embodiments of the composition have been shown and disclosed herein above, those of skill in the art will recognize certain modifications, permutations, additions, and sub-combinations thereof. It is therefore intended that the consumable shall be interpreted to include all such modifications, permutations, additions, and sub-combinations as are within their true spirit and scope, and no limitations are intended to the details of construction or design herein shown.

What is claimed is:

1. A composition for treating skin of a patient, the composition comprising:
a biofilm neutralizer including a sugar alcohol;
an antifungal including grapefruit seed extract; and
a skin soother including aloe vera and an oil, the oil only having a fatty acid chain length of less than C12 or greater than C24.

2. The composition of claim 1 wherein the sugar alcohol is Xylitol.

3. The composition of claim 1 wherein the biofilm neutralizer further includes colloidal silver.

4. The composition of claim 1 wherein the antifungal further includes at least one of grapefruit seed extract, sea salt, tea tree oil, and colloidal silver.

5. The composition of claim 1 wherein the skin soother further includes at least one of methylsulfonylmethane, niacinamide, hyaluronic acid, and urea.

6. The composition of claim 1 wherein the oil includes an essential oil.

7. The composition of claim 1 wherein the skin soother further includes Allantoin.

8. The composition of claim 1 further comprising purified water comprising greater than approximately 50 percent of the overall weight and less than 60 percent of the overall weight.

9. A composition for treating skin of a patient treatment, the composition having an overall weight, the composition comprising:
a biofilm neutralizer including a sugar alcohol, the sugar alcohol including Xylitol comprising greater than approximately 0.001 percent of the overall weight and less than 90.0 percent of the overall weight;
an antifungal including grapefruit seed extract and colloidal silver, and
a skin soother including aloe vera, methylsulfonylmethane, and an oil, the oil only having a fatty acid chain length of less than C12 or greater than C24, the aloe vera comprising greater than approximately 0.001 percent of the overall weight and less than 90.0 percent of the overall weight.

10. The composition of claim 9 wherein the antifungal further includes at least one of sea salt and an essential oil.

11. The composition of claim 9 wherein the skin soother further includes at least one of niacinamide, hyaluronic acid, and urea.

12. The composition of claim 9 wherein the oil includes an essential oil.

13. The composition of claim 9 wherein the skin soother further includes Allantoin.

14. The composition of claim 9 further comprising purified water comprising greater than approximately 50 percent of the overall weight and less than 60 percent of the overall weight.

15. The composition of claim 9 wherein the colloidal silver comprises greater than approximately 7 percent of the overall weight and less than 12 percent of the overall weight.

16. The composition of claim 9 wherein the methylsulfonylmethane comprises greater than approximately 4 percent of the overall weight and less than 7 percent of the overall weight.

17. The composition of claim 9 wherein the grapefruit seed extract comprises greater than approximately 0.1 percent of the overall weight and less than 2.0 percent of the overall weight.

18. The composition of claim 9 wherein the oil comprises greater than approximately 0.01 percent of the overall weight and less than 0.11 percent of the overall weight.

19. A method for manufacturing the composition of claim 9.

20. A composition for treating skin of a patient, the composition comprising:
a biofilm neutralizer including Xylitol and colloidal silver;
an antifungal including grapefruit seed extract, sea salt, and tea tree oil; and
a skin soother including aloe vera, methylsulfonylmethane, niacinamide, hyaluronic acid, rosemary essential oil, and urea, the oils only having a fatty acid chain length of less than C12 or greater than C24.
